SEQUENCE LISTING (2 sequences)

SEQ ID NO: 1 - DNA, 20 bp, Artificial Sequence
  artificially synthesized primer sequence
agcgaaccagtatcgagaac

SEQ ID NO: 2 - DNA, 20 bp, Artificial Sequence
  artificially synthesized primer sequence
ttacagaaccacactcggac

(19) United States
(12) Patent Application Publication
(10) Pub. No.: US 2011/0256627 A1
(43) Pub. Date: Oct. 20, 2011

(54) Cutting device for culturing the next generation of cells

(75) Inventors: Shao-Yin Chen, Hsinchu (TW); Shiaw-Min Hwang, Hsinchu (TW)

(73) Assignee: Food Industry Research and Development Institute, Hsinchu (TW)

(21) Appl. No.: 12/926,941

(22) Filed: Dec. 20, 2010

(30) Foreign Application Priority Data
Dec. 25, 2009 (TW) 98224454

(51) Int. Cl. C12N 5/0735; C12M 1/00

(52) U.S. Cl. 435/379; 435/283.1

(57) ABSTRACT

The present invention is related to a device for subculturing embryonic stem cells, which mainly contains a cutting brush having a handle and a brush stem. The brush stem has a stem and a plurality of discrete upright wires disposed on the stem. The brush stem is mounted onto a front end of the handle, and an angle between the brush stem and the handle ranges from 70 to 90 degrees. The distance between two adjacent wires is smaller than the diameter of a block of the embryonic stem cells to be cut for next generation culturing, so that the cutting brush can evenly cut the block into a plurality of smaller blocks of embryonic stem cells.

### CROSS REFERENCE TO RELATED PATENT APPLICATIONS

The present patent application claims the benefit of Taiwan Patent Application Number 98224454 filed Dec. 25, 2009.

### FIELD OF THE INVENTION

The present invention relates to a cutting device for subculturing cells being applicable in next generation culturing of embryonic stem cells (such as human embryonic stem cells).

### DESCRIPTION OF PRIOR ART

The human embryonic stem cells can differentiate into a variety of tissue cells in the human body, and is poised to play a key role in revolutionizing the current medical applications. The success of its related clinical applications is essentially hinged on the quantity and quality of cell culture used for laboratory tests, and thus improvements in culturing embryonic stem cells are crucial to clinical treatments, basic researches, and private pharmaceutical industries. Unlike most cells in culture, human embryonic stem cells are passaged as clumps. Single cell dissociation only decreases cell survival and doesn't contribute to successful subculture. Accordingly, known methods for cutting the cells include: (1) mechanical passage by glass pipet or rollers. It is time consuming, and the materials accompanying the use of rollers is highly expensive, and not environment-friendly. (2) enzyme passage by collagenase, trypsin or dispase. It is problematic in that the stability of chromosomes is potentially affected.

The inventor of the present invention has been working in the stem cell bank in Taiwan, and fully understands the negative effects the aforesaid shortcomings have on the research of the human embryonic stem cells; this invention was tested repeatedly and proposed in response to the shortcomings.

### SUMMARY OF THE INVENTION

An object of the present invention is to provide a cutting device for subculturing cells being applicable in next generation culturing of embryonic stem cells (such as human embryonic stem cells), which does not have the shortcomings of the aforesaid methods.

Another object of the present invention is to provide a cutting device for subculturing cells that can be sterilized and re-used.

To achieve the above-mentioned objects of the invention, a cutting device for subculturing cells has been disclosed according to this invention, which includes a cutting brush, and the cutting brush comprises:

a handle comprising a holding portion for being held by human hands, and an inserting portion disposed at a front end of the holding portion;

a brush stem comprising a stem and a plurality of discrete upright wires disposed on the stem, and the wires may be disposed as one row or multiple parallel rows;

wherein the brush stem is mounted onto the inserting portion, and an angle between the brush stem and the holding portion ranges from 70 to 90 degrees.

Preferably, the cutting device of the invention further comprises a petri dish, and a length of the brush stem having a plurality of upright wires is smaller than a diameter of the petri dish.

Preferably, a distance between and two adjacent wires of the plurality of upright wires is smaller than 100 μm. More preferably, the distance between any two adjacent wires is greater than 20 μm.

Preferably, the angle between the holding portion and the inserting portion of the handle is adjustable. More preferably, an end of the inserting portion away from the brush stem is pivotally joined with a front end of the holding portion.

Preferably, the angle between the holding portion and the inserting portion of the handle is fixed and cannot be changed. More preferably, the holding portion and the inserting portion are formed as a unibody.

Preferably, the inserting portion has a mounting hole, and the brush stem is inserted and joined thereinto.

Preferably, the holding portion and the inserting portion are formed linearly, and an angle between an insertion direction of the mounting hole and the inserting portion ranges from 70 to 90 degrees.

Preferably, an angle between the holding portion and the inserting portion ranges from 70 to 90 degrees, and an insertion direction of the mounting hole is parallel to the inserting portion.

Preferably, each of the wires is of a diameter ranging from 0.1-10 μm, and a length ranging from 0.05-5 mm.

Preferably, the stem of the brush stem is Z-shaped, wherein the Z-shaped stem has a first horizontal portion inserted into and joined with the mounting hole, and a second horizontal portion having the plurality of discrete upright wires disposed thereon; a front end of the first horizontal portion is joined to a rear end of the second horizontal portion by a vertical or arc portion.

The present invention also discloses a method for cutting blocks of embryonic stem cells (such as human embryonic stem cells) for subculturing by using the cutting device of the invention, comprising the step of using the plurality of discrete upright wires to horizontally slice through a block of embryonic stem cells (such as human embryonic stem cells) in a petri dish intended for subculturing, thereby dividing the block of embryonic stem cells into a plurality of smaller blocks of embryonic stem cells.

### BRIEF DESCRIPTION OF DRAWINGS

FIG. 1 is a schematic side view showing a cutting device for subculturing cells according to a first preferred embodiment of this invention.

FIG. 2 is a schematic side view showing a cutting brush for subculturing cells according to a second preferred embodiment of this invention.

FIG. 3 is a schematic side view showing a cutting brush for subculturing cells according to a third preferred embodiment of this invention.

FIG. 4 is a schematic side view showing a cutting device for subculturing cells according to a fourth preferred embodiment of this invention.

FIG. 5 contains photographs showing the colonies of embryonic stem cells using conventional passage tool (control group) and the cutting device (cutter group) of the present invention (3 passages; cultured for 12 days) (40× magnification; scale bar 100 um).

FIG. 6 is a plot depicting the degrees of undifferentiation of embryonic stem cells by using an alkaline phosphatase staining for the control group and the cutter group.

FIGS. 7A and 7B are photographs depicting the expression of Oct-4 RNA transcripts, an undifferentiation marker of embryonic stem cells (3 passages) by performing a reverse transcription polymerase chain reaction and an agarose gel electrophoresis (A); and a fluorescence staining (B).

FIG. 8 contains photographs showing the formation of embryoid body (EB) measuring a tridermic differentiation for the control group and the cutter group.

### DETAILED DESCRIPTION OF PREFERRED EMBODIMENTS

The invention relates to a device for subculturing embryonic stem cells, and more particularly to a device for subculturing human embryonic stem cells. The device mainly comprises a cutting brush having a handle and a brush stem. The handle is preferably a metal bar, and can be sterilized under high temperature and high pressure for reuse. The handle may also be disposable, and is preferably made from reusable materials, such as by thermoplastic injection molding. An end of the handle has a mounting hole disposed thereon, which allows the brush stem to be inserted and secured thereinto. The brush stem can either be secured into the mounting hole by the friction there between, or directly combined with an end of the handle by the aforesaid thermoplastic injection molding. Preferably, the brush stem has a stem made of metal and wires made of gamma-ray resistant materials (such as nylon), or the stem and wires of the stem brush can all be made of gamma-ray sterilization resistant materials. According to a preferred embodiment of the invention, a brush stem of adequate length can be used accordingly to work with petri dishes of different diameters, so as to facilitate operations of experiments. The adequate ratios between petri dish diameters and adequate brush stem lengths are listed as follows:

| Petri Dish | Petri Dish Diameter | Brush Stem Length |
|---|---|---|
| 6 cm petri dish | 6 cm | 3 cm |
| 6 well plate | 4.8 cm | 2.4 cm |
| 24 well plate | 2.4 cm | 1.2 cm |

* Brush stem length refers to the part of brush stem that has wires.

The distances between the wires of the brush stem is smaller than the diameter of a block of (human) embryonic stem cells to be cut for next generation culture, so as to evenly cut the block into a plurality of smaller blocks of embryonic stem cells. For instance, a block of human embryonic stem cells is ready for subculture when reaching 500 μm in diameter on average, therefore the distances between the wires of the brush stem should be smaller than 100 μm.

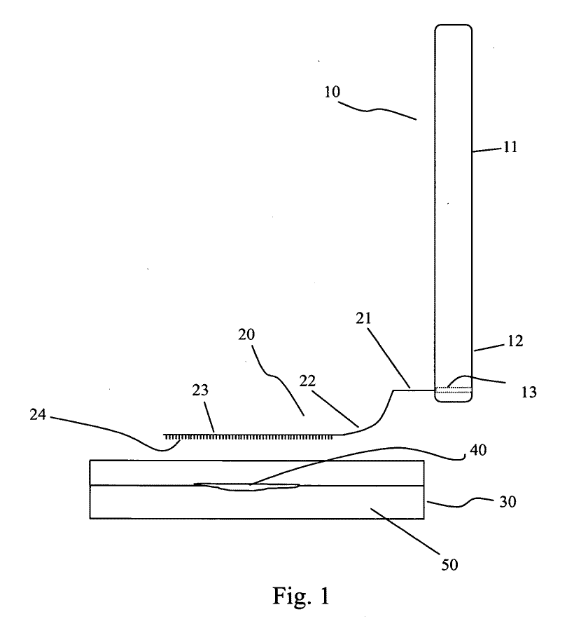

A cutting device for subculturing cells according to a first preferred embodiment of this invention is shown in FIG. 1, which comprises a cutting brush and a petri dish 30. The cutting brush includes a handle 10 and a brush stem 20. The brush stem 20 has a stem and a plurality of discrete upright wires 24 disposed on the stem, wherein the stem is bent into a first horizontal portion 21, a second horizontal portion 23, and an arc portion 22 joining the first horizontal portion and the second horizontal portion together. The wires 24 are fixedly disposed on the second horizontal portion 23 of the brush stem at even distances from each other.

The handle 10 is comprised of a holding portion 11 for being held by human hands, and an inserting portion 12 disposed at a front end of the holding portion. The inserting portion 12 further includes a mounting hole 13 (shown as dotted lines). The first horizontal portion 21 of the brush stem is inserted and secured into the mounting hole 13. An angle between the second horizontal portion 23 of the brush stem and the holding portion 11 is approximately 90 degrees.

The petri dish 30 has a cell culture medium 50 and a block of embryonic stem cells 40 cultured in the cell culture medium therein. When operating, an operator holds the holding portion 11 by hand, and horizontally slices the plurality of discrete upright wires 24 through the block of embryonic stem cells 40, and then rotates the petri dish by 90 degrees before slicing horizontally through the block again, such that the block is cut into a plurality of smaller blocks of embryonic stem cells.

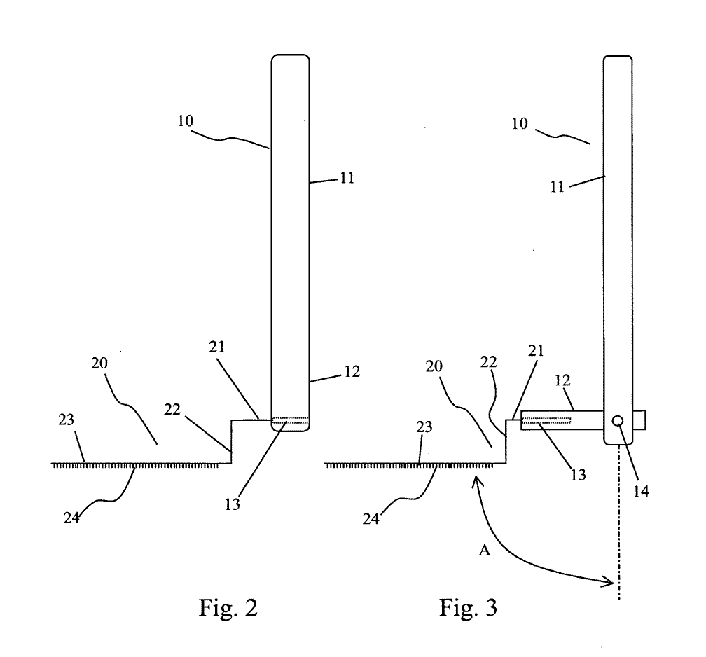

A cutting brush for subculturing cells according to a second preferred embodiment of this invention is shown in FIG. 2, wherein the components (parts) similar to the cutting device in FIG. 1 are marked with the same numbers. The cutting brush of the second preferred embodiment of the invention is structurally similar to the cutting device shown in FIG. 1, except that a vertical portion 22 joins the first horizontal portion 21 and the second horizontal portion 23 together in the bent stem.

A cutting brush for subculturing cells according to a third preferred embodiment of this invention is shown in FIG. 3, wherein the components (parts) similar to the cutting brush in FIG. 2 are marked with the same numbers. Except for the handle 10, the cutting brush of the third preferred embodiment of the invention is structurally similar to the one shown in FIG. 2. FIG. 3 shows that the holding portion 11 of the handle 10 can be rotatingly joined with the inserting portion 12 by using a joining pin 14, such that an angle between the holding portion 11 and the inserting portion 12 can be adjusted. An angle A between the second horizontal portion 23 of the brush stem and the holding portion 11 can be adjusted to approximately 70 to 90 degrees.

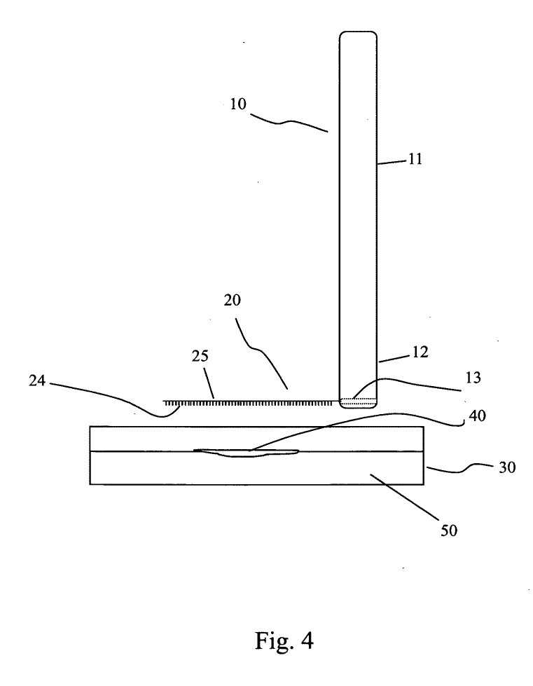

A cutting device for subculturing cells according to a fourth preferred embodiment of this invention is shown in FIG. 4, wherein the components (parts) similar to the cutting device in FIG. 1 are marked with the same numbers. The cutting device for culturing the next generation of cells according to the fourth preferred embodiment of the invention is structurally identical to the one shown in FIG. 1, except that the cutting brush has a horizontal stem 25.

### Human ES Cells Culture and Embryoid Body (EB) Formation

To test the efficacy of this innovative cell cutter, we split embryonic stem (ES) cell cultures into two groups. For the control groups, the ES cells were undergone conventional passage treatment. ES cells were grown on MEF feeders and maintained in DMEM/F12 (supplemented with 20% knockout serum replacement, 2 mM L-glutamine, 1% non-essential amino acids, 4 ng/ml human bFGF (all from Invitrogen), and 0.1 mM 2-mercaptoethanol) (Sigma). Culture medium was changed daily and subculture was performed every 4-6 days by 1 mg/ml collagenase IV (Invitrogen). After PBS washes, the cells were dispersed by repeated pipeting and re-plated onto freshly thawed MEF. For the cutter group, ES cells were treated under the same condition as the control group except that the cutting device of the present invention was used to replace repeated pipeting. All cells were maintained in a 5% CO₂-humidified atmosphere at 37° C.

To test the pluripotency of ES cells in culture, we performed EB formation assay. ES cells were detached and dispersed into small clumps. The cell clumps were then cultured in bacterial-graded Petri dish (Corning Inc) for up to 7 days in the presence of DMEM supplemented with 10% FBS (Invitrogen). All cells were maintained in a 5% CO₂-humidified atmosphere at 37° C.

### RNA Extraction and RT-PCR

Total RNA was isolated using the mirVana miRNA isolation kit (Invitrogen) according to the manufacturer's instructions. RNA quality and quantity were evaluated on a NanoDrop spectrophotometer. One microliter of cDNA sample was PCR amplified with gene-specific primers (Forward sequence: AGC GAA CCA GTA TCG AGA AC, Reverse sequence: TTA CAG AAC CAC ACT CGG AC) by using optimized PCR cycles to obtain amplified reactions in a linear range. GAPDH was used for the internal control reaction on the same sample.

### Alkaline Phosphatase Staining & Immunofluorescence Assay

ES cells at post passage day five were removed from the incubator and then the medium was withdrawn before been fixed with 4% paraformaldehyde (Sigma) for 5 min at room temperature. Alkaline phosphatase staining was carried out with an alkaline phosphatase staining kit (Millipore, SCR004) according to the manufacturer's protocol. For the immunofluorescence assay, we grew ES cells on feeders in 24-well plate until ready. After removing media, we fixed ES cells with 4% paraformaldehyde for 15 min at room temperature. Cells were washed with 0.5% Triton x-100/PBS, at room temperature for 15 minutes and then blocked with 5% goat serum for 1 hour at room temperature. Primary antibodies against Oct-4 (Abcam) were diluted in PBS at 1:400 dilutions and then reacted with ES cells for 1 hour, at 37° C. Washed the cells with PBS, 5 minutes each, 3 times at room temperature (RT). Fluorescein anti-rabbit IgG were dilated with PBS at 1:5000 dilutions (Counter stain with DAPI/PBS), 1 hour at 37° C. before microscopic observation.

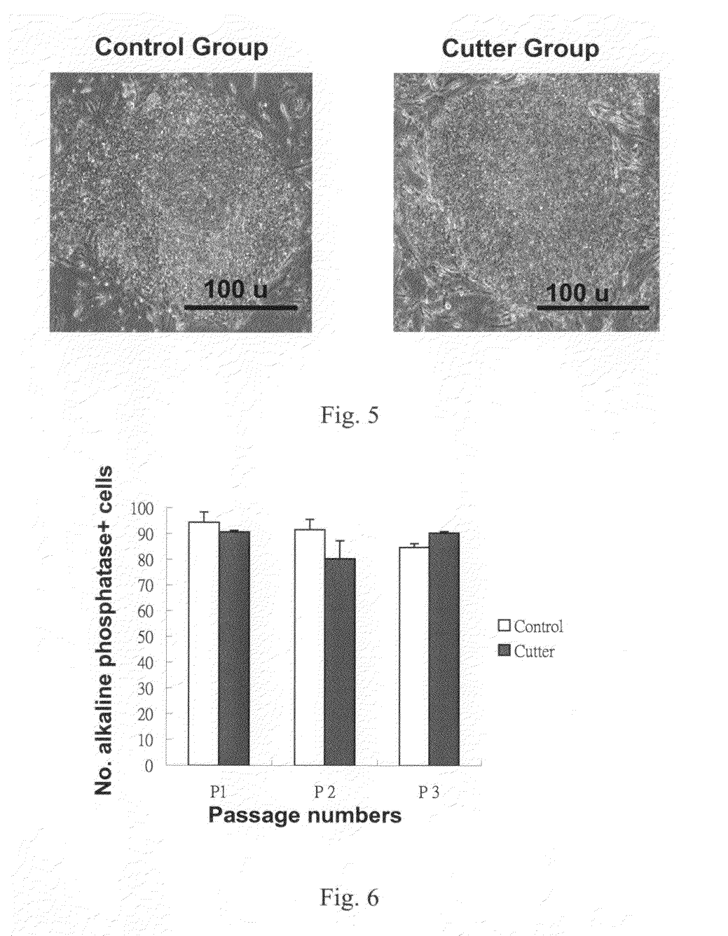

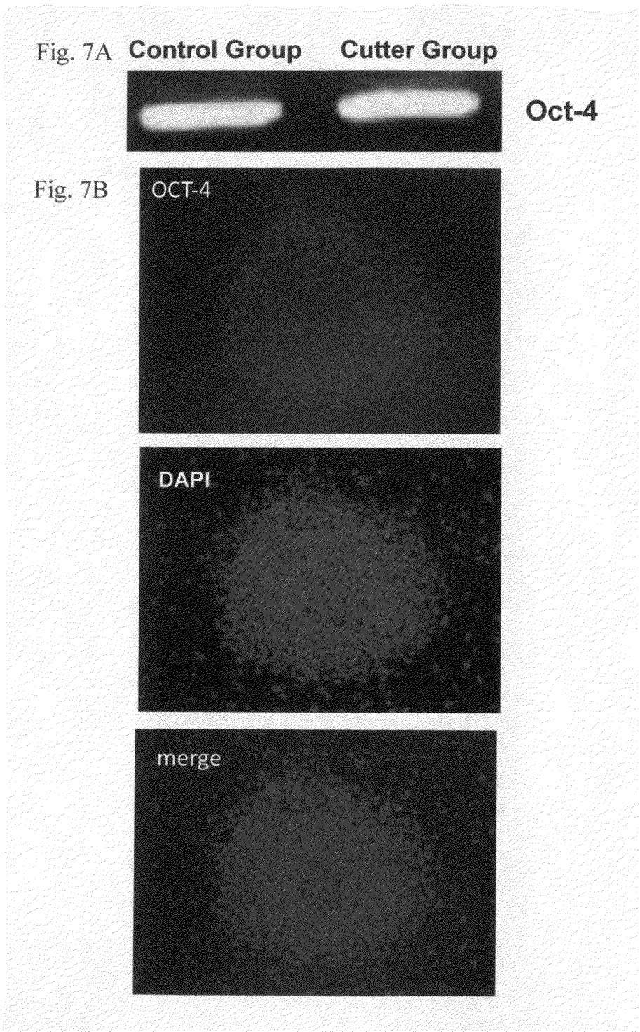

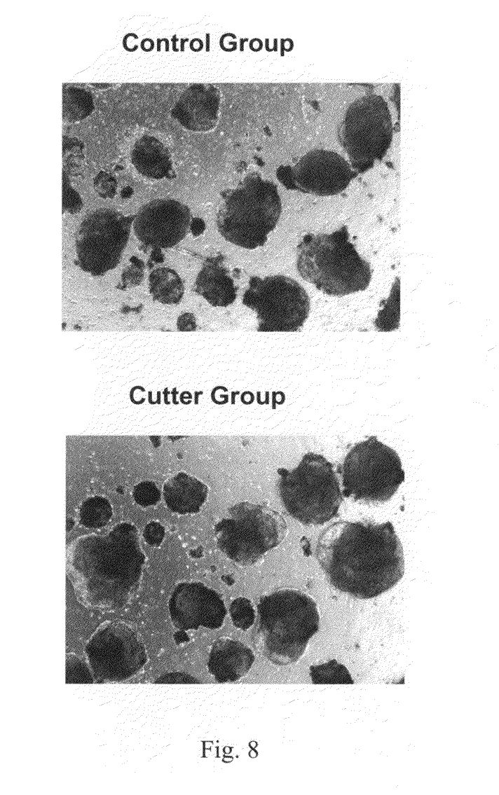

In order to identify whether human embryonic stem cells cultured through the conventional passage tool and the cutter passage tool of the present invention remain indifferent or not, they were observed by a phase contrast microscope. The results are shown in FIG. 5. Besides, human embryonic stem cells monitored by using the alkaline phosphatase staining are shown in FIG. 6; monitored in Oct-4 marker expression by using the reverse transcription PCR is shown in FIG. 7A; and by using the fluorescence staining is shown in FIG. 7B. In the assay to form embryoid body (EB), we know the embryonic stem cells passage by the cutter can be induced to differentiate into three lineages of the cells, as shown in FIG. 8.

While preferred embodiments of the invention are described above, it will be apparent to one skilled in the art that various changes and modifications may be made therein without departing from the spirit and scope of the invention. Accordingly, the invention is not limited except by the appended claims.

What is claimed is:

1. A cutting device for subculturing cells, having a cutting brush, and the cutting brush comprising:
a handle having a holding portion for being held by human hands, and an inserting portion disposed at a front end of the holding portion; and
a brush stem having a stem and a plurality of discrete upright wires disposed on the stem, the wires being disposed as one row or multiple parallel rows;
wherein the brush stem is mounted onto the inserting portion, and an angle between the brush stem and the holding portion ranges from 70 to 90 degrees.

2. The cutting device of claim 1, further comprising a petri dish, and a length of the brush stem having a plurality of upright wires is smaller than a diameter of the petri dish.

3. The cutting devices of claim 2, wherein a distance between any two adjacent wires of the plurality of upright wires is smaller than 100 μm.

4. The cutting device of claim 3, wherein the distance between any two adjacent wires is greater than 20 μm.

5. The cutting devices of claim 2, wherein an angle between the holding portion and the inserting portion of the handle is adjustable.

6. The cutting device of claim 5, wherein an end of the inserting portion away from the brush stem is pivotally joined with a front end of the holding portion.

7. The cutting devices of claim 2, wherein an angle between the holding portion and the inserting portion of the handle is fixed and cannot be changed.

8. The cutting device of claim 7, wherein the holding portion and the inserting portion are formed as a unibody.

9. The cutting devices of claim 2, wherein the inserting portion has a mounting hole, and the brush stem is inserted and joined thereinto.

10. The cutting device of claim 9, wherein the holding portion and the inserting portion are formed linearly, and an angle between an insertion direction of the mounting hole and the inserting portion ranges from 70 to 90 degrees.

11. The cutting device of claim 9, wherein an angle between the holding portion and the inserting portion ranges from 70 to 90 degrees, and an insertion direction of the mounting hole is parallel to the inserting portion.

12. The cutting devices of claim 2, wherein each of the wires is of a diameter ranging from 0.1-10 μm, and a length ranging from 0.05-5 mm.

13. The cutting device of claim 9, wherein the stem of the brush stem is Z-shaped, in which the Z-shaped stem has a first horizontal portion inserted into and joined with the mounting hole, and a second horizontal portion having the plurality of discrete upright wires disposed thereon, and a front end of the first horizontal portion is joined to a rear end of the second horizontal portion by a vertical or arc portion.

14. A method for cutting blocks of embryonic stem cells for subculture by using the cutting device set forth in claim 1, comprising using the plurality of discrete upright wires to horizontally slice through a block of embryonic stem cells in a petri dish intended for subculture, thereby dividing the block of embryonic stem cells into a plurality of smaller blocks of embryonic stem cells.
